SEQUENCE LISTING (9 sequences)

SEQ ID NO: 1 - protein, 9 aa, Artificial Sequence
  segment capable of recognizing and interacting with beta-secretase
SEVNLDAEF

SEQ ID NO: 2 - protein, 14 aa, Artificial Sequence
  peptide substrates employed in assay for beta-secretase
TEEISEVNLDAEFK

SEQ ID NO: 3 - protein, 14 aa, Artificial Sequence
  peptide substrates employed in assay for beta-secretase
TEEISEVNLDAEFK

SEQ ID NO: 4 - protein, 14 aa, Artificial Sequence
  peptide substrates employed in assay for beta-secretase
TEEISEVNLDAEFK

SEQ ID NO: 5 - protein, 15 aa, Artificial Sequence
  peptide substrates employed in assay for beta-secretase
TKKISEVNLDAEFRK

SEQ ID NO: 6 - protein, 12 aa, Artificial Sequence
  statine-derived peptide inhibitor of beta-secretase
KTEISEVNVAEF

SEQ ID NO: 7 - protein, 4 aa, Artificial Sequence
  bioconjugate structure
DEVD

SEQ ID NO: 8 - protein, 4 aa, Artificial Sequence
  bioconjugate structure
DEVD

SEQ ID NO: 9 - protein, 4 aa, Artificial Sequence
  segment capable of being cleaved by a caspase enzyme
DEVD

(19) United States
(12) Patent Application Publication
(10) Pub. No.: US 2005/0014160 A1
(43) Pub. Date: Jan. 20, 2005

(54) Assays for protease enzyme activity

(76) Inventors: Sriram Kumaraswamy, Santa Fe, NM (US); Komandoor Achyuthan, Santa Fe, NM (US); Xiaobo Shi, Los Alamos, NM (US); Troy Bergstedt, Cochiti, NM (US); Frauke Rininsland, Santa Fe, NM (US); Duncan McBranch, Santa Fe, NM (US); David Whitten, Albuquerque, NM (US)

Correspondence Address: Supervisor, Patent Prosecution Services;Piper Rudnick LLP, 1200 Nineteenth Street, N.W., Washington, DC, 20036-2412, US

(21) Appl. No.: 10/621,311

(22) Filed: Jul. 18, 2003

(51) Int. Cl. C12Q 1/68; G01N 33/53; C07H 21/04

(52) U.S. Cl. 435/6; 435/7.5; 530/391.1; 536/25.32

(57) ABSTRACT

Bioconjugates, kits and assays for detecting the activity of protease enzymes such as β-secretase and caspase enzymes are described. The bioconjugates include a tether having a segment capable of recognizing and interacting with (e.g., being cleaved by) the enzyme, a fluorescer including a plurality of fluorescent species conjugated to a first location on the tether, and a quencher conjugated to a second location on the tether. The segment capable of recognizing and interacting with the protease enzyme (e.g., β-secretase or caspase) is located between the first and second locations on the tether. The plurality of fluorescent species are associated with one another such that the quencher is capable of amplified super-quenching of the fluorescer. The assay is suitable for screening potential drugs for their efficiency in inhibiting the activity of protease enzymes such as β-secretase in a high throughput format where the potential drugs are evaluated.

This application is related to U.S. patent application Ser. No. 10/226,300, filed Aug. 23, 2002, pending, which is incorporated herein by reference in its entirety.

### BACKGROUND

1. Technical Field

The present application relates generally to assays for the detection of molecular interactions. In particular, the present application relates to bioconjugates that can be used to detect the activity of protease enzymes (e.g., β-secretase and caspase enzymes), kits including the bioconjugates and assays involving the use of the bioconjugates to detect enzyme activity.

2. Discussion of the Background

Protease enzymes play a key role in cellular biology and have become priority targets for new pharmaceuticals. The interest and importance for the measurement of proteolytic enzyme activity is rapidly increasing both for research, drug discovery and development. Apoptosis is a remarkable process responsible for cell death in development, normal tissue turnover and also accounts for many cell deaths following exposure to cytotoxic compounds, hypoxia or viral infection. Many cancer therapeutic agents exert their effects through initiation of apoptosis. Even the process of carcinogenesis seems sometimes to depend upon a selective, critical failure of apoptosis that permits the survival of mutagenic DNA damage. Apoptosis is suspected to contribute to chronic degenerative processes such as Parkinsons's disease and heart failure. Several caspase enzymes are thought to mediate very early stages of apoptosis. As caspase enzymes are probably the most important effector molecules for triggering the biochemical events which lead to apoptotic cell death, assays for determination of caspase activity can detect apoptosis earlier than many other commonly used methods.

Alzheimer's disease is characterized by the extracellular deposition of insoluble amyloid plaques, which consist of 4 kD amyloid β-peptide (Aβ). Glenner et al., Biochem. Biophys. Res. Commun. 1984, 120, pp. 885-890. Aβ derives from proteolysis of the amyloid precursor protein (APP) by the β and γ secretases to create the N and C-termini of Aβ respectively. Kang et al., Nature 1987, 325, 733-736. The enzyme β-secretase has been shown to be essential for nerve cells to form the plaques. Vassar et. al., Science 1999, 286, 735-741.

Most current therapeutic approaches to Alzheimer's disease involve finding drugs that block the β-secretase enzyme's catalytic site and prevent its function. Thus, it is of high importance to develop rapid and sensitive assay platforms that are compatible with high throughput screening to facilitate the discovery of new drugs that will combat the disease.

Therefore, there exists a need to rapidly and accurately detect and quantify the activity of protease enzymes such as β-secretase and caspase enzymes with high sensitivity.

### SUMMARY

According to a first aspect of the invention, a bioconjugate is provided which includes: a tether comprising a segment capable of recognizing and interacting with β-secretase; a fluorescer comprising a plurality of fluorescent species conjugated to a first location on the tether; and a quencher conjugated to a second location on the tether. According to this aspect of the invention, the segment capable of recognizing and interacting with β-secretase is located between the first and second locations on the tether. The plurality of fluorescent species are associated with one another such that the quencher is capable of amplified super-quenching of the fluorescer. The segment capable of recognizing and interacting with β-secretase can comprise the peptide sequence: SEVNLDAEF (SEQ ID NO:1).

According to a second aspect of the invention, a method of assaying for β-secretase activity in a sample is provided including: incubating the sample with a bioconjugate as set forth above; and measuring the fluorescence of the incubated sample. The measured fluorescence of the incubated sample is an indication of the presence and/or the amount of β-secretase activity in the sample. The method may further include: measuring the fluorescence of a control and comparing the fluorescence of the control to the fluorescence of the incubated sample wherein a difference in the fluorescence between the control and the incubated sample is an indication of the presence or amount of β-secretase activity in the sample. The sample can include β-secretase and a test compound and the method can be an assay for the ability of the test compound to inhibit β-secretase activity. The fluorescer can comprise a solid support wherein the plurality of fluorescent species are associated with the solid support.

According to a third aspect of the invention, a method of assaying for β-secretase activity of a sample is provided including: incubating the sample with a bioconjugate comprising a quencher and a ligand conjugated to a tether at first and second locations respectively, wherein the tether comprises a segment between the first and second locations capable of recognizing and interacting with β-secretase; adding a fluorescer to the incubated sample to form a sample mixture, the fluorescer comprising a solid support associated with a plurality of fluorescent species, wherein the solid support comprises a moiety capable of binding the ligand of the bioconjugate such that the bioconjugate can bind to the solid support and wherein binding of the bioconjugate to the solid support results in amplified superquenching of the fluorescer; allowing the ligand on the bioconjugate to bind to the solid support; and subsequently measuring the fluorescence of the sample mixture. The amount of fluorescence of the sample mixture indicates the presence and/or amount of β-secretase activity in the sample. The ligand can be a biotin moiety and the moiety on the solid support can be an avidin, neutravidin or streptavidin moiety. The method can further include: adding the fluorescer to a second sample that contains the bioconjugate but has not been incubated with the enzyme to form a control; measuring the fluorescence of the control; and comparing the fluorescence of the control to the fluorescence of the sample mixture; wherein a difference in the fluorescence between the control and the sample mixture is an indication of the presence and/or the amount of β-secretase in the sample. The sample can comprise β-secretase and a test compound and the method can further include: incubating a second sample containing no test compound with the bioconjugate; adding the fluorescer to the incubated second sample to form a control; measuring the fluorescence of the control; and comparing the fluorescence of the control to the fluorescence of the sample mixture, wherein the difference in the fluorescence between the control and the sample mixture is an indication of the ability of the test compound to inhibit β-secretase activity in the sample.

According to a fourth aspect of the invention, a kit is provided including: a fluorescer comprising a plurality of fluorescent species associated with a solid support; and a bioconjugate comprising a quencher and a ligand conjugated to a tether at first and second locations respectively, wherein the tether comprises a segment between the first and second locations capable of recognizing and interacting with β-secretase. The solid support comprises a moiety capable of binding the ligand on the bioconjugate and the plurality of fluorescent species are associated with one another such that the quencher is capable of amplified superquenching of the fluorescer when the ligand of the bioconjugate is bound to the solid support. The ligand can be a biotin moiety and the moiety capable of binding the ligand can be selected from the group consisting of avidin, neutravidin and streptavidin. The segment capable of recognizing and interacting with β-secretase can be the peptide sequence:

| | |
|---|---|
| SEVNLDAEF. | (SEQ ID NO:1) |

According to a fifth aspect of the invention, a bioconjugate is provided including: a tether comprising a segment capable of recognizing and interacting with β-secretase; a quencher conjugated to a first location on the tether, the quencher capable of quenching the fluorescence of a fluorescer comprising a plurality of associated fluorescent species; and a biotin molecule conjugated to a second location on the quencher; wherein the segment capable of recognizing and interacting with the target biomolecule is located between the first and second locations on the tether and wherein the plurality of fluorescent species are associated with one another such that the quencher is capable of amplified quenching of the fluorescer. The segment capable of recognizing and interacting with β-secretase can comprise the peptide sequence: SEVNLDAEF (SEQ ID NO:1).

According to a sixth aspect of the invention, a method for assaying for target enzyme activity in a sample is provided which includes: incubating the sample with a bioconjugate comprising a quencher conjugated to a tether, wherein the tether comprises a segment capable of being cleaved by the target enzyme; adding a fluorescer to the incubated sample to form a sample mixture, the fluorescer comprising a plurality of fluorescent species associated with one another such that association of the fluorescer with the quencher results in amplified superquenching of the fluorescer; and allowing the target enzyme to cleave the tether, wherein cleavage of the tether results in a quencher containing fragment that has a greater tendency to associate with the fluorescer than the bioconjugate; and subsequently measuring fluorescence of the sample mixture. The amount of fluorescence of the sample mixture indicates the presence and/or amount of target enzyme activity in the sample. The association between the quencher and fluorescer can be the result of coulombic attraction, hydrogen bonding forces, van der waals forces, or covalent bond formation. For example, the fluorescer and the bioconjugate can each have an overall negative charge and the quencher containing fragment can have a net positive charge. The fluorescer can be an anionic conjugated polymer and the quencher can be a cationic electron or energy transfer quencher. According to one embodiment, the bioconjugate is represented by the following formula:

| | |
|---|---|
| D-E-V-D-QSY7′. | (SEQ ID NO:7) |

According to a further embodiment, the fluorescer is a virtual polymer comprising an aggregate of donor cyanine dyes and the quencher is an acceptor cyanine dye which, when conjugated to the tether, is unable to form an aggregate with the donor cyanine dyes. The inability of the bioconjugate to form an aggregate with the donor cyanine dyes can be the result of charge effects or steric effects. The acceptor cyanine dye can be a fluorescent molecule or a non-fluorescent molecule. When the acceptor cyanine dye is a fluorescent molecule, the fluorescence of the acceptor can be measured. Alternatively, the fluorescence of the donor can be measured. The assay can be an intracellular assay or an extracellular assay.

According to a seventh aspect of the invention, a method for assaying for target enzyme activity in a sample is provided which includes: incubating the sample with a bioconjugate comprising a fluorescent dye conjugated to a tether, wherein the tether comprises a segment which can be cleaved by the target enzyme to produce a fluorescent dye containing fragment, and wherein the fluorescent dye containing fragment is capable of forming a dye aggregate which has a different absorption spectra than the bioconjugate; allowing the enzyme to cleave the bioconjugate; and measuring the fluorescence of the sample mixture by exciting the sample at a wavelength wherein the dye aggregate absorbs to a greater extent than the bioconjugate. The amount of fluorescence of the sample mixture indicates the presence and/or amount of target enzyme activity in the sample. The fluorescent dye can be a cyanine molecule. The fluorescent dye containing fragment released from the bioconjugate by enzyme cleavage can be capable of forming a J-aggregate. The target enzyme can be a caspase enzyme. For example, the target enzyme can be caspase-3 and the bioconjugate can have a structure represented by the following formula:

| | |
|---|---|
| D-E-V-D-Cyanine. | (SEQ ID NO:8) |

According to an eighth aspect of the invention, a kit is provided which includes a fluorescer comprising a plurality of fluorescent species and a bioconjugate comprising a quencher conjugated to a tether, wherein the tether comprises a segment capable of being cleaved by a caspase enzyme. According to this embodiment of the invention, cleavage of the tether results in a quencher containing bioconjugate fragment that has a greater tendency to associate with the fluorescer than the bioconjugate and association of the fluorescer with the quencher results in amplified superquenching of the fluorescer. The plurality of associated fluorescent species can be associated with a solid support. The target caspase enzyme can be caspase-3 and the segment capable of being cleaved by a caspase enzyme can be the peptide sequence:

| | |
|---|---|
| DEVD. | (SEQ ID NO:9) |

The quencher containing bioconjugate fragment can associate with the fluorescer via coulombic attraction, hydrogen bonding forces, van der waals forces, or covalent bond formation. For example, the fluorescer and the bioconjugate can each have an overall negative charge and the quencher containing bioconjugate fragment can have a net positive charge.

A kit as set forth above is also provided wherein the fluorescer is a virtual polymer comprising an aggregate of donor cyanine dyes and the quencher is an acceptor cyanine dye and wherein the acceptor, when conjugated to the tether, is unable to form an aggregate with the donor cyanine dyes. The inability of the bioconjugate to form an aggregate with the donor can be the result of charge effects or steric effects. The acceptor cyanine dye can be a fluorescent molecule or a non-fluorescent molecule.

According to a ninth aspect of the invention, a bioconjugate is provided which includes a tether comprising a segment capable of being cleaved by a caspase enzyme and a quencher conjugated to the tether. The caspase enzyme can be caspase-3 and the segment capable of being cleaved by a caspase enzyme can be the peptide sequence:

| | |
|---|---|
| DEVD. | (SEQ ID NO:9) |

The quencher can be a cationic electron or energy transfer quencher.

According to a tenth aspect of the invention, a bioconjugate is provided which includes a fluorescent dye conjugated to a tether wherein the tether comprises a segment which can be cleaved by the target enzyme to produce a fluorescent dye containing fragment. The fluorescent dye containing fragment is capable of forming a dye aggregate which has a different absorption spectra than the bioconjugate. The target enzyme can be a caspase enzyme such as caspase-3. The fluorescent dye can be a cyanine dye. According to a further embodiment, the fluorescent dye containing fragment is capable of forming a J-aggregate. The segment capable of being cleaved by the target enzyme can be the peptide sequence:

| | |
|---|---|
| DEVD. | (SEQ ID NO:9) |

### BRIEF DESCRIPTION OF THE DRAWINGS

FIG. 1 is an illustration of two general schemes for conducting assays for protease (e.g., β-secretase) enzyme activity.

FIG. 2 is an illustration of a quencher-tether (QT) assay for Caspase-3 enzyme activity.

FIG. 3 is an illustration of a J-aggregate assay format for Caspase-3 activity.

### DETAILED DESCRIPTION OF EMBODIMENTS

The quencher-tether-ligand (QTL) approach to biosensing takes advantage of the superquenching of fluorescers such as fluorescent polyelectrolytes by electron transfer and energy transfer quenchers. In one format, the fluorescer (e.g., fluorescent polymer) P is co-located on the surface of a solid support such as a polymer microsphere along with a receptor for a specific analyte. The receptor can be attached to a solid support (e.g., a bead support) by, for example, a covalent linkage or a biotin-biotin binding protein (BBP) association. The assay is based on competition for the receptor between the analyte and a synthetic QTL conjugate. While the fluorescence of the polymer-receptor ensemble is unaffected by the binding of the analyte, it is quenched when the QTL is bound. Quantitative assays for small molecules and proteins have been demonstrated using this technology. Chen et al., Proc. Nat. Acad. Sci. 1999, 96, 12287-12292.

U.S. patent application Ser. No. 10/226,300, filed Aug. 23, 2002, discloses a sensor for protease enzymes which includes a reactive tether linking a fluorescer (e.g., a fluorescent polymer) P with the quencher Q. This modification of the QTL approach is referred to as “QTP”, where the QTP ensemble is a reactive molecular sensor that includes a quencher, Q, linked via a peptide tether that is recognized and cleaved by the target enzyme, to a fluorescer P. In the absence of a specific association of, or reaction of the QTP molecule with an enzyme or other target molecule, the fluorescence of P is attenuated or completely quenched by the relative close proximity of Q. When the tether T is recognized and cleaved by the target, separation of the Q and P components is accomplished such that the fluorescence of the latter is turned on. Since the enzyme-induced cleavage of T is catalytic, amplification of the detection event occurs and thus affords detection of the enzyme at very low levels.

In a different format, a QTB molecule is used wherein “B” refers to a biotin group. The appended biotin binds to the biotin binding protein (BBP) that is co-located with the polymer in the sensor and facilitates efficient quenching of the polymer by the quencher. When the peptide is cleaved by target enzyme, the quencher and biotin groups are separated from each other and thus there is no quenching of the polymer fluorescence.

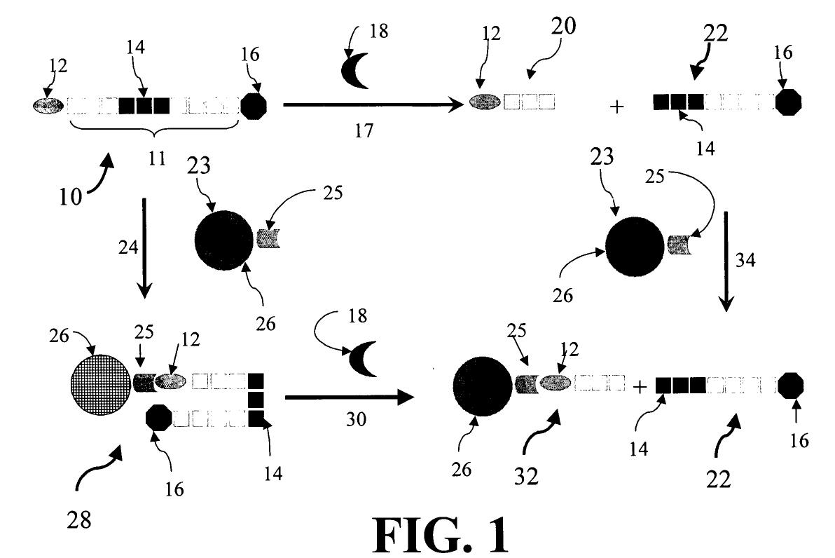

Two general schemes for conducting protease assays are illustrated in FIG. 1. In the first scheme shown in FIG. 1 which is represented by arrows 17 and 34, a bioconjugate (10) comprising a quencher (16), a biotin moiety (12) and a tether (11) linking the quencher (16) and biotin moiety (12) is incubated (17) with a protease (e.g., β-secretase) enzyme (18). The tether comprises a recognition sequence (14) capable of being recognized (e.g., cleaved) by the β-secretase enzyme (18). The incubated bioconjugate is then contacted (34) with a fluorescer comprising a biotin binding protein (23). A fluorescent polymer coated microsphere (26) having streptavidin groups (25) on the surface thereof is shown in FIG. 1 as the fluorescer (23). When the biotin moiety (12) of the uncleaved bioconjugate (10) reacts with the streptavidin moiety (25) on the fluorescent polymer coated microsphere (26), the fluorescence from the fluorescer (23) is quenched as shown by quenched bioconjugate (28). However, as shown in the first scheme for protease assays, when the biotin moiety of the biotin containing fragment (32) resulting from the cleavage of the tether by the enzyme (18) reacts with the streptavidin moiety (25) on the microsphere (26), the fluorescence from the fluorescer (23) is not quenched. Therefore, the fluorescence can be used to determine the presence and or the amount of β-secretase enzyme activity.

In the second scheme for protease assays shown in FIG. 1, which is represented by arrows 24 and 30, a bioconjugate (10) comprising a quencher (16), a biotin moiety (12) and a tether (11) linking the quencher (16) and biotin moiety (12) is first contacted (24) with a fluorescer comprising a biotin binding protein (23). The tether (11) comprises a recognition sequence (14) capable of being recognized (e.g., cleaved) by the β-secretase enzyme (18). When the biotin moiety (12) of the uncleaved bioconjugate (10) reacts with the streptavidin moiety (25) on the fluorescent polymer coated microsphere (26), the fluorescence from the fluorescer (23) is quenched as shown by quenched bioconjugate (28). The resulting quenched bioconjugate (28) is then incubated (30) with a protease (e.g., β-secretase) enzyme (18). Cleavage of the tether by the enzyme (18) results in separation of the quencher containing fragment (22) from the fluorescer containing fragment (32). As a result, the fluorescence from the fluorescer increases (i.e., the amount of quenching of the fluorescer is reduced).

The peptide substrates employed in the QTB assay are tri-functional in that they comprise a peptide sequence in the middle that can be recognized and cleaved by a target enzyme, a biotin functional group on one end that facilitates the binding of the QTB to the polymer-receptor ensemble and the quencher that efficiently quenches polymer fluorescence when it is brought in close proximity to the polymer.

The β-secretase enzyme has been shown to recognize and bind the following peptide sequence:

| | |
|---|---|
| SEVNLDAEF. | (SEQ ID NO:1) |

Cai et al., Science 1993, 259, 514-516. After binding, the enzyme cleaves the peptide bond between leucine and aspartic acid. According to one embodiment of the invention, a QTB peptide substrate for β-secretase comprises a tether including this sequence which is flanked by biotin on one end and a quencher on the other.

Structures of exemplary peptide substrates that can be employed in the assay for β-secretase are listed below:

| | |
|---|---|
| (QSY-7)-TEEISEVNLDAEFK-(Nε- | (SEQ ID NO:2) |
| Biotin); | |
| (QSY-7)-TEEISEVNLDAEFK-(Nε-PEG- | (SEQ ID NO:3) |
| Biotin); | |
| (AZO)-TEEISEVNLDAEFK-(Nε-Biotin); | (SEQ ID NO:4) |
| and | |
| (AZO)-TKKISEVNLDAEFRK-(Nε-Biotin); | (SEQ ID NO:5) |

wherein QSY-7, AZO, Biotin and PEG-Biotin are represented by the following structures: 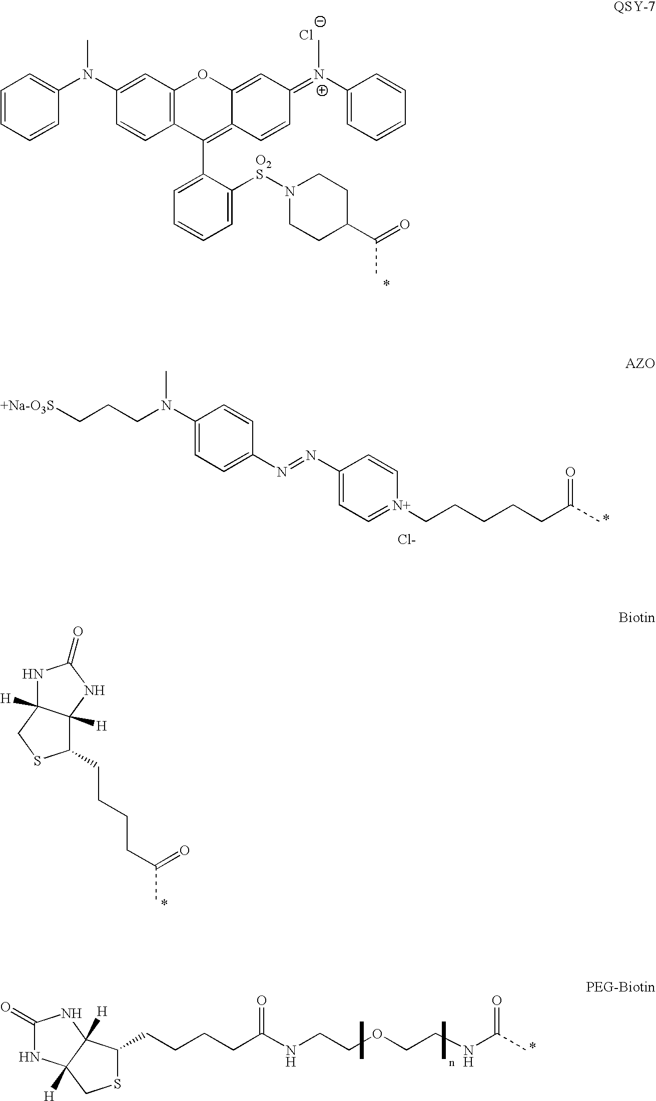
and wherein “*” denotes the point of attachment of each moiety to the polypeptide tether and “Nε” denotes linkage of the biotin moiety to the lysine residue of the polypeptide tether through the E-amino group of the lysine residue (K). The QSY-7 and AZO moieties as shown above are attached to the polypeptide tether through the free amino group of the threonine residue.

Thus when β-secretase cleaves the peptide tether, biotin will remain on one of the resulting fragments while the quencher is physically separated from it and remains on the other fragment. The quencher is thus left without a biotin moiety to help bind the fluorescer-receptor ensemble. Consequently, the biotin containing fragment of the cleaved substrates should not quench the fluorescence of the ensemble.

The biotin is specifically included in the QTB bioconjugate to bring the polymer and quencher together by binding a BBP of the polymer-BBP ensemble. Although the interaction between biotin and a BBP is disclosed above, the biotin-BBP interaction can easily be replaced with any system that is capable of uniting the fluorescer and the quencher. For example, the uniting interaction can be any biological antigen-receptor combination, or, a metal-ligand binding event, or, a chemical reaction between two or more reacting species. The interaction can include but is not limited to, either hydrogen bonding or coulombic attraction or covalent bonding.

The quencher, Q, is designed to absorb the radiative energy from the excited polymer to quench the fluorescence. Exemplary quenchers include, but are not limited to, the following species: neutral, positively or negatively charged or zwitter-ionic, non-fluorescent or fluorescent, organic, inorganic, organometallic, biological or polymeric, or energy or electron-transfer species. According to one embodiment of the invention, the quencher is a non-fluorescent small molecule dye such as QSY-7 or Azo dye. According to one embodiment, the quencher is capable of amplified quenching or super-quenching of the plurality of fluorescent species of the fluorescer. According to a further embodiment, the quencher is capable of re-emitting as fluorescence the absorbed radiative energy from the fluorescer.

According to an embodiment of the invention, the tether of the bioconjugate will comprise the peptide sequence:

| | |
|---|---|
| SEVNLDAEF. | (SEQ ID NO:1) |

This sequence may be flanked on either side by more amino acids or other chemical and biological entities. The length of the QTB tether is not critical to the assay. According to an embodiment of the invention, the quencher is within approximately 100 Å of the polymer when the QTB is bound to the polymer-receptor ensemble. This spacing can be achieved even for very long QTB tethers since they are usually present in some secondary folded conformation that is likely to bring the quencher close to the polymer.

The fluorescer (F) comprises a plurality of fluorescent species. According to an exemplary embodiment, the fluorescer is a conjugated polymer that can be either neutral or positively or negatively charged or zwitter-ionic. The fluorescer (F) may also be a side-chain polymer comprising a non-conjugated backbone with pendant fluorescent dyes that exhibit J-type aggregation behavior. The fluorescer may also comprise a plurality of independent small molecule fluorescent chromophores that aggregate on a solid surface to form “virtual” polymers.

Structures of exemplary fluorescent polymers (1) and (2) are given below:

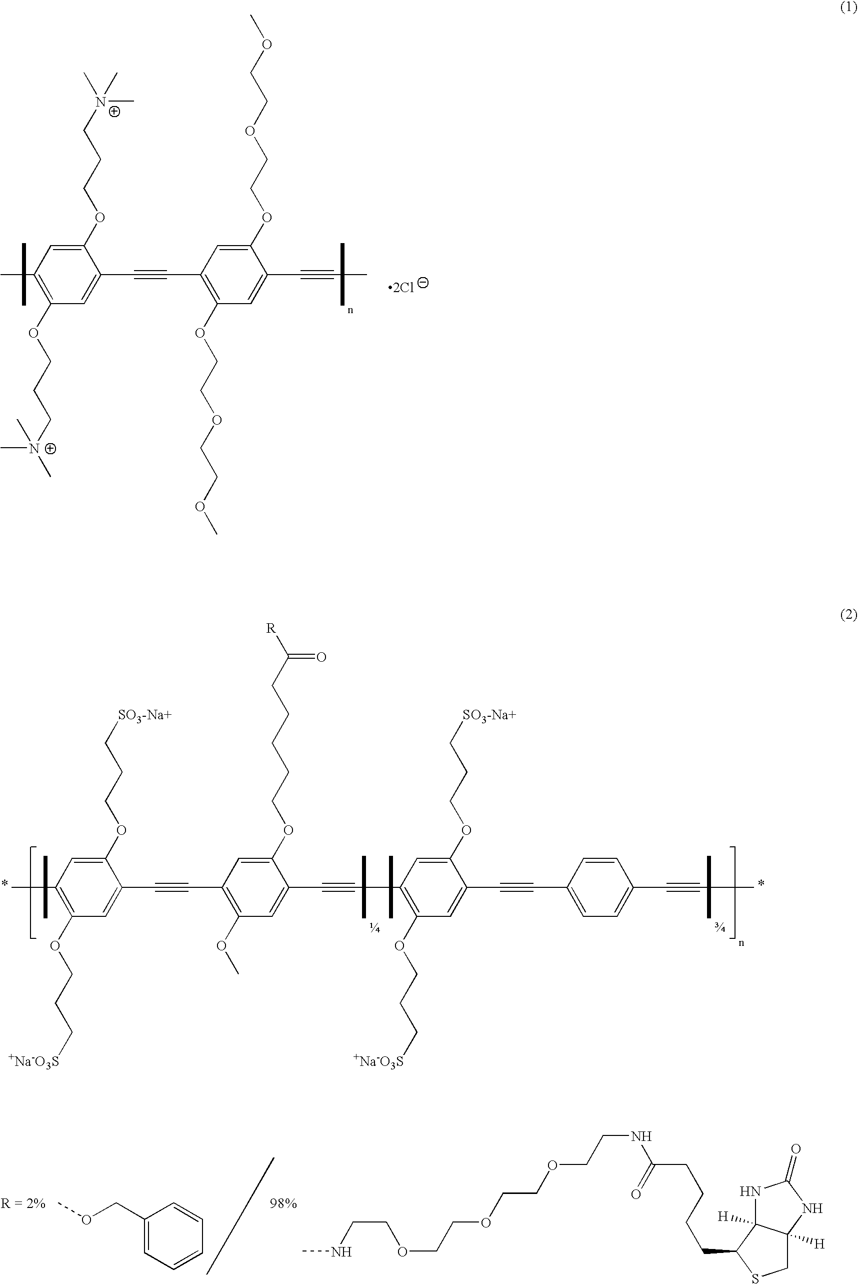

According to one embodiment of the invention, the fluorescent polymer is co-located with a biotin binding protein (BBP) either in solution or anchored to a solid-support. In one embodiment, a positively charged PPE polymer as set forth in formula (1) above is adsorptively coated onto neutravidin-functionalized anionic carboxylic acid-bound latex microspheres having a diameter of 0.6 μm (Polymer Ensemble A). In another embodiment, a biotinylated anionic PPE polymer represented by formula (2) above is complexed to avidin to form a solution sensor ensemble (Polymer Ensemble B). The polymer binds avidin through the biotin-avidin interaction to form cross-linked supra-molecular ensembles that comprise free biotin binding sites available for the QTB bioconjugate. Their quench behavior can be optimized by varying the ratios of polymer (2) and BBP in the mixture.

In other embodiments of the invention, each of these polymer formats can be improved by adding Biotinylated R-Phycoerythrin (BRPE). The resulting formats are denoted Polymer Ensembles C and D. In the presence of the BRPE dopant, the excited polymer chromophores transfer their energy to the nearby BRPE molecules, which then re-emit that energy more efficiently to provide sharp, red-shifted fluorescent signal. The fluorescence of the BRPE is then quenched when the QTB binds.

In another embodiment of the invention, the fluorescent polymer is a biological ensemble comprising phycoerythrin or phycobilisome. These proteins, which consist of a polymeric ensemble containing ˜34 chromophores which harvest energy and collect it at a moderately protected emitting site, are some of the most fluorescent entities known, and when conjugated to biotin binding protein, they serve as excellent sensors in the QTP assay for β-secretase and other proteases (Polymer ensemble E). For example, a 1:1 covalent Streptavidin-B-Phycoerythrin conjugate (SAv-BPE) exhibits superquenching in the presence of the β-secretase peptide substrate represented by SEQ ID NO:2, as demonstrated by a Stem-Volmer quenching constant of ˜1×10⁸ M⁻¹ for a 2.5 nM solution of the fluorescent conjugate.

The QTP assay for β-secretase has been optimized using the various polymer formats and peptide substrates revealed in this document. The amplification of detection sensitivity afforded by the light harvesting property of the polymer combined with the super-quenching efficiency of the quencher make the QTP assay significantly more sensitive than other fluorescence based assays.

In one embodiment of the sensor, the QTB is incubated with the polymer-receptor ensembles to form the QTP unit. The assay for β-secretase is then performed by exposing the sample containing the enzyme to QTP and following the “recovery” of fluorescence in a continuous monitoring format. The QTP unit has little or no fluorescence when the quencher is in close proximity to the polymer. Depending on the activity of enzyme present in the sample, cleavage of peptide substrate occurs, leading to a reduction in the quench response, or, in effect increased fluorescence in the sample. In another embodiment of the sensor, the QTB entity is exposed to the β-secretase enzyme-containing sample and incubated at CRT for a short period of time. After incubation, the polymer-receptor ensemble is added to the sample and the fluorescence intensity of the final mixture measured. By comparing the fluorescence so measured to that of a sample containing the same amount of QTB and polymer without enzyme, a measure of the fluorescence increase attributable to enzyme activity alone is obtained. The incubation of enzyme and substrate is performed in a buffer solution (Assay Buffer) that has been optimized to provide maximum activity of enzyme against the QTB substrate. The polymer is usually made up in a buffer solution (Polymer Buffer) that has been optimized to perform the twin tasks of stopping the reaction upon addition to the reaction mixture and to provide maximal quench response from the polymer-QTB interaction. In a current embodiment of the sensor, the QTL assay is capable of detecting β-secretase activity in solutions of concentrations less than 1 nM in 30 minutes.

In another embodiment, the QTL assay is capable of detecting the inhibition of β-secretase activity in unknown samples. Whereas, in the absence of any inhibitory substance in the reaction mixture, the enzyme would cleave a large portion of the reactive tether present, in the presence of an effective inhibitor, the enzyme loses most of its activity. The QTL assay provides evidence of partial or total inhibition of enzyme activity through a lowering or complete lack of “fluorescence recovery” in such samples.

Exemplary fluorescers include a polymer or oligomer comprising a plurality of fluorescent repeating units or a solid support associated with a plurality of fluorescent species. When a solid support is used, one or more quenchers can each be linked to the solid support through a reactive tether. Exemplary solid supports include, but are not limited to, the following: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes. The fluorescer can be selected from the group consisting of: conjugated polyelectrolytes; biotinylated conjugated polyelectrolytes; functionalized conjugated oligomers; charged conjugated polymers; uncharged conjugated polymers; conjugated polymer blends; and J-aggregated polymer assembly comprising assembled monomers or oligomers. For example, the fluorescer can be a poly(L-lysine) polymer or oligomer having cyanine pendant groups. The fluorescer can also be a virtual polymer. Alternatively, the fluorescer can be constructed from an oligosaccharide.

The fluorescent polymer or oligomer can be associated with a solid support by covalent attachment to the solid support, adsorption onto the surface of the solid support, or by interactions between a biotin moiety on the fluorescent polymer or oligomer and an avidin, neutravidin or streptavidin moiety on the solid support surface.

The fluorescer can be conjugated to the tether via a protein molecule. Exemplary protein molecules include avidin, neutravidin, and streptavidin.

In one experiment, the well-known statine derived peptide inhibitor of β-secretase called STA-200 was shown to provide IC₅₀ value in the range of nanomolar concentrations when the sample was incubated for just fifteen minutes. The structure of the statine-derived peptide inhibitor of β-secretase is given below:

| | |
|---|---|
| KTEISEVN-(Sta)-VAEF-OH. | (SEQ ID NO:6) |

Wherein “Sta” represents a statine residue. In a current embodiment of the invention, the assays are performed in the wells of microwell plates such as a 96-well or a 384-well plate. The assay is thus convenient for use in a conventional microplate reader available in most drug screening laboratories. The assay is homogeneous, sensitive and rapid for the detection of β-secretase enzyme activity. In the current embodiment of the invention, the QTL assay for β-secretase is tolerant of the presence of DMSO up to the extent of 10% in the reaction mixture. The assay is tolerant of up to 10% of the presence of methanol and acetonitrile in the reaction mixtures. The assay is thus suitable for screening of potential drugs in a high-throughput format where the potential drugs are evaluated for their efficiency in inhibiting the activity of β-secretase against the peptide substrate. In the current embodiment of the invention, the assay is highly robust and provides Z′-values of upwards of 0.6 at approximately 10% conversion of the peptide substrate.

According to a further embodiment of the invention, a method for assaying for target enzyme activity in a sample is provided which includes: incubating the sample with a bioconjugate comprising a quencher conjugated to a tether, wherein the tether comprises a segment capable of recognizing and interacting with the target enzyme; adding a fluorescer to the incubated sample to form a sample mixture, the fluorescer comprising a plurality of fluorescent species associated with one another such that a association of the fluorescer with the quencher results in amplified superquenching of the fluorescer; allowing the target enzyme to cleave the bioconjugate and release the quencher; and subsequently measuring the fluorescence of the sample mixture. The amount of fluorescence of the sample mixture indicates the presence and/or amount of the target enzyme activity in the sample.

The above assay uses a quencher-tether (QT) bioconjugate that is unable to interact with the added fluorescer to quench its fluorescence. Upon cleavage by the target enzyme, the quencher is released from the bioconjugate, thus enabling its interaction with the fluorescer to cause a fluorescence quench response. The interaction between the quencher and fluorescer can be the result of coulombic attraction, hydrogen bonding forces, van der waals forces, or covalent bond formation.

According to one embodiment of the invention, the fluorescer is an anionic conjugated polymer and the quencher is a cationic electron or energy transfer quencher. According to this embodiment, the quencher-tether bioconjugate has an overall negative charge and hence does not interact with and quench the fluorescer. Upon cleavage of the bioconjugate by the target enzyme, however, the cationic quencher is released which enables it to quench the fluorescer.

In a further embodiment of the invention, the fluorescer is a “virtual polymer” comprising an aggregate of donor cyanine dyes and the quencher is an acceptor cyanine dye. According to this embodiment, when the acceptor is conjugated to the tether, it is unable to participate in an aggregate with donor cyanine dyes and hence does not quench donor fluorescence. The inability of the bioconjugate to form an aggregate with the donor can be the result of charge effects, steric effects or a combination thereof. When the tether is cleaved by the target enzyme, however, the quencher containing fragment of the bioconjugate is able to participate in the aggregate with donor cyanine dyes and, as a result, quenches the donor fluorescence. The acceptor cyanine dye can be either a fluorescent or a non-fluorescent molecule. If the acceptor cyanine is itself fluorescent, its participation in the aggregate with the donor cyanine will result in sensitized fluorescence from the acceptor. The sample can thus be monitored for enzymatic activity by following either the decrease in fluorescence intensity of the donor or the increase in signal intensity of the acceptor. An assay according to this embodiment is capable of enzyme activity determination in both intra- and extracellular assay formats.

According to a further aspect of the invention, a method for assaying for target enzyme activity in an intracellular or extracellular sample is provided which includes: incubating the sample with a bioconjugate comprising a fluorescent dye conjugated to a tether wherein the fluorescent dye when cleaved from the bioconjugate by an enzyme is capable of forming aggregates such as J-type aggregates, and the tether comprises a segment capable of recognizing and interacting with the enzyme; allowing the enzyme to cleave the bioconjugate and release the dye; and subsequently measuring the fluorescence of the sample mixture by exciting the sample at a wavelength wherein the dye aggregate would absorb but not the dye labeled bioconjugate. The amount of fluorescence of the sample mixture indicates the presence and/or amount of the target enzyme activity in the sample.

According to this embodiment of the invention, the fluorescent dye can be a cyanine molecule which when conjugated to the tether exists as the monomer and has broad absorption and emission spectra. Upon cleavage of the tether by the target enzyme, the fluorescent dye containing fragment is released from the bioconjugate, and is capable of forming weakly bound aggregates such as a J-aggregate which exhibit large shifts of the absorption and emission maxima to longer wavelengths and much narrower spectra in comparison to the monomer. Therefore, monitoring for emission from the sample by exciting it at the absorption maximum for the aggregate will result in low or no signal from the bioconjugate itself. In the presence of dye aggregates formed by free dye molecules obtained upon enzymatic cleavage of the bioconjugate, the sample will provide a signal whose intensity is an indicator of enzyme activity.

Cyanine dyes capable of forming J-Aggregates are disclosed in Lu et al., “Surface Enhanced Superquenching of Cyanine Dyes as J-Aggregates on Laponite Clay Nanoparticles”, Langmuir, Vol. 18, No. 20, pp. 7706-7713 (2002). This reference also discloses acceptor cyanine dyes capable of quenching the fluorescence of J-aggregated donor cyanine dyes. J-Aggregate polymers are disclosed in Lu et al., “Superquenching in Cyanine Pendant Poly(L-lysine) Dyes: Dependence on Molecular Weight, Solvent, and Aggregation”, J. Am. Chem. Soc., Vol. 124, No. 3 (2002).

Experimental

Generalized Assay Procedure for the Determination of β-Secretase Activity

In a 384-well plate, 5 mL of β-secretase peptide substrate solution of appropriate concentration was mixed with a given amount of β-secretase enzyme in a total volume of 10 mL in assay buffer. In the same plate, a control experiment was also set up where the same amount of peptide was taken in assay buffer but no enzyme was added. The experiments were usually done in triplicate sets for both sample and control. After a given time period of incubation at CRT, the fluorescent polymer in polymer buffer was added to the sample and control wells. The well plate was shaken inside the plate reader and the fluorescence intensity of the samples was measured. The difference in relative fluorescence units between sample and control wells is a measure of enzyme activity.

In the above example, the polymer can include any of the following:
A. Biotin binding protein (BBP) functionalized polystyrene latex carboxylic acid microspheres that are coated with conjugated polymer 1;
B. Solution complex of BBP and biotinylated polymer 2;
C. Microspheres as in A that are doped with a small amount of Phycoerythrin or related fluorescent protein conjugated to biotin;
D. Solution Sensor as in B doped with a small amount of Phycoerythrin or related fluorescent protein conjugated to biotin; and
E. Covalent conjugate of Phycoetythrin or another fluorescent protein and BBP.

The peptide substrate in the above experiments can be any of those listed above. All of these peptides contain an amino acid sequence that is recognized and cleaved by β-secretase enzyme. In addition, each of the substrates can be tagged with a biotinyl group on one end and with a quencher molecule on the other end.

The following examples illustrate the ability of the QTP assay using the various polymer formats and the various peptides to determine the activity of β-secretase in a sample, to demonstrate inhibition of enzyme activity in the presence of a known inhibitor of β-secretase, and prove the robustness of the assay in the presence of various potential interferents such as organic solvents, colored compounds, fluorescent compounds, commonly found proteins, surfactants and positively and negatively charged ions at their physiological concentrations.

### EXAMPLE 1

To 5 μL of a 400 nM solution of BSEC-1 in assay buffer in the well of a 384-well plate, 20 ng of β-secretase enzyme in 5 μL of assay buffer was added. In the control well, 5 μL of the 400 nM BSEC-1 solution was mixed with 5 μL of assay buffer alone without any enzyme. The mixtures were incubated at CRT for 30 minutes. At the end of the incubation period, an 18.5 μL suspension of polymer A containing 1×10⁷ microspheres was added to each well. The plate was shaken inside the microplate reader for 60 seconds, then the samples were probed for emission at 530 nm by exciting them at 420 nm. A 475 nm cut-off filter was used for the measurements. Measurements of the sample and control were each performed in triplicate and the results averaged to provide reliable data. The relative fluorescence units (RFU) obtained for the control was 5529±286 while the sample gave an RFU of 9045±109. The gain in RFU of the sample over the control (also called Delta) is directly proportional to the β-secretase activity in the sample.

### EXAMPLE 2

When BSEC-2 peptide containing the azo dye quencher was used in the experiment, enhanced assay performance was seen. To 5 μL of a 400 nM solution of BSEC-3 was added a 5 μL solution of β-secretase (60 ng). Control wells were set up here just as in Example 1. The enzyme and control wells were incubated for only ten minutes at CRT before the addition of polymer. The polymer suspension added to each well contained 1×10⁷ microspheres in 20 μL. The samples were probed for fluorescence intensity at 530 nm by exciting them at 440 nm and using the 475 nm cut-off filter. The RFU obtained for the control was 8111±707 while the sample gave an RFU of 10996±424.

### EXAMPLE 3

The assay performance was further improved when the polymer microsphere sample A was doped with a small amount of Biotin-R-Phycoerythrin (Biotin-R-PE) conjugate. In one experiment, BSEC-1, BSEC-3 (a,b) (same peptide substrate synthesized by different vendors) and BSEC-4 were all exposed to β-secretase enzyme under identical conditions to compare their efficiencies in the assay. Polymer sample C was prepared fresh just before the start of experiment by mixing together Polymer A and Biotin-R-PE in a ratio designed to provide 5×10⁶ microspheres doped with 100 fmol of Biotin-R-PE per well in a volume of 10 μL.

To 5 μL of a 300 nM solution of each peptide substrate in assay buffer in separate wells was added 10 ng of β-secretase enzyme in 5 μL of assay buffer to start the reactions. Each reaction was performed in triplicate. Controls were performed for each peptide without enzyme in quadruplicate. The samples were incubated at CRT for 60 minutes. At the end of the incubation period, the Biotin-R-PE doped polymer C was added to each well. The plate was shaken in the plate reader for 60 seconds, then probed for emission at 576 nm by exciting at 440 nm and using 475 nm cut-off filter. BSEC-1 gave RFU values of 6270±196 for control and 9645±152 for sample. BSEC-3a gave RFUs of 7656±20 for control and 15022±743 for sample while BSEC-3b gave RFUs of 6054±41 and 12265±913 for control and sample respectively. In comparison, BSEC-4 gave values of 9207±364 and 9732±319 for control and sample respectively.

### EXAMPLE 4

The polymer solution sensor B was prepared by mixing together 56.5 nmol of Avidin (Biotin binding protein, BBP) and 848 nmol of biotinylated PPE polymer 2 in a total volume of 11.3 μL and incubating at CRT for 24 hours. The polymer and the BBP combine with each other through the biotin-avidin interaction to form stable entities. The solution sensor thus prepared was diluted appropriately with polymer buffer at the beginning of each experiment.

### EXAMPLE 5

To 5 μL of a 400 nM solution of BSEC-1 in assay buffer in a 384 well plate was added 30 ng of β-secretase dissolved in 5 μL of assay buffer. The mixture was made in triplicate and incubated for 30 minutes at CRT. The control wells contained only peptide and no enzyme. After incubation, a 100-fold dilution of the above solution sensor was added at 20 μL to each well. The plate was shaken inside the microplate reader and the wells were probed by exciting the polymer at 440 nm and measuring emission intensity at 530 run. The control wells gave an average RFU value of 5400±200 and the sample wells containing enzyme gave RFU of 8350±200.

### EXAMPLE 6

The assay performance was much improved by doping the solution sensor polymer B with a small amount of Biotin-R-PE. Polymer sensor D was made at the beginning of each experiment by incubating a 200-fold dilution of the master stock of Polymer B with Biotin-R-PE in a ratio that would provide 250 fmol of the latter in 40 μL of the mixture. To 5 μL of a 300 nM solution of BSEC-3 in assay buffer was added 30 ng of β-secretase enzyme in 5 μL of assay buffer. After incubating the the control and sample mixtures for 30 minutes at CRT, 40 μL of the doped solution sensor D was added to each well. The plate was shaken inside the plate reader for 60 seconds and the wells were probed for fluorescence intensity by exciting the polymer at 440 nm and measuring the emission at 576 nm using a 475 nm cut-off filter. The control wells gave an average RFU value of 5200±100 while the sample wells gave a corresponding value of 14500±200.

### EXAMPLE 7

Highly fluorescent proteins containing multiple chromophores such as Phycoerythrin can be employed independently in the QTL assay for β-secretase. To 5 μL of a 300 nM solution of BSEC-1 in assay buffer was added 20 ng of β-secretase in 5 μL of assay buffer. The control wells had only BSEC-1 and no enzyme. After incubating the mixtures for 30 minutes, 10 μL of a 5 nM (50 fmol) solution of the Streptavidin-B-Phycoerythrin conjugate (SAv-BPE, Polymer E) was added to each well. The plate was shaken inside the plate reader and the fluorescence intensities of the sample and control wells were measured at 576 nm by exciting at 490 nm and using 515 nm cut-off filter. The control wells gave an average RFU of 7671±286 while the enzyme wells gave a corresponding value of 11828±556.

When the assay was performed under identical conditions with BSEC-3 instead of BSEC-1, the delta RFU was better: RFU of control was 11639±335 and RFU of sample was 18032±228.

### EXAMPLE 8

STA-200 is a well-known inhibitor of β-secretase activity. To 2.5 μL of an 800 nM solution of BSEC-1 in assay buffer was added 30 ng of β-secretase and the volume made up to a total of 10 μL with assay buffer. In another well, 2.5 μL of 800 nM BSEC-1 solution was incubated with a pre-mixed solution of 2.5 μL of STA-200 solution and 30 ng of β-secretase so that the final volume is again 10 μL. Two control reactions were also set up in separate wells where one had only peptide in assay buffer while the other had peptide and inhibitor incubating in assay buffer. All samples were incubated at CRT for 15 minutes followed by the addition to each of 19.2 μL of Polymer A suspension (1×10⁷ microspheres) in polymer buffer. The mixtures were then probed for emission at 530 nm by exciting them at 440 nm and using 475 nm cut-off filter. The mixture of peptide and enzyme alone gave a delta-over-control RFU of 4468±85. In comparison, a mixture of peptide, enzyme and 60 nM STA-200 (600 fmol) gave a delta-over-control RFU of 2746±241 while a mixture containing 250 nM STA-200 (2.5 pmol) gave a corresponding value of only 865±178.

Peptide Substrates, Kits and Assays for Other Protease Enzyme

Peptide substrates, kits and assays for β-secretase are disclosed above. Following is a discussion of exemplary peptide substrates that can be used in assays for other protease enzymes such as caspase enzymes. Specific substrates for caspase-3 enzyme activity are also described below.

In particular, a method for assaying for target enzyme activity in a sample is provided which includes: incubating the sample with a bioconjugate comprising a quencher conjugated to a tether, wherein the tether comprises a segment capable of being cleaved by the target enzyme; adding a fluorescer to the incubated sample to form a sample mixture, the fluorescer comprising a plurality of fluorescent species associated with one another such that association of the fluorescer with the quencher results in amplified superquenching of the fluorescer; and allowing the target enzyme to cleave the tether, wherein cleavage of the tether results in a quencher containing fragment that has a greater tendency to associate with the fluorescer than the bioconjugate; and subsequently measuring fluorescence of the sample mixture. The amount of fluorescence of the sample mixture indicates the presence and/or amount of target enzyme activity in the sample. The association between the quencher and fluorescer can be the result of coulombic attraction, hydrogen bonding forces, van der waals forces, or covalent bond formation. For example, the fluorescer and the bioconjugate can each have an overall negative charge and the quencher containing fragment can have a net positive charge. The fluorescer can be an anionic conjugated polymer and the quencher can be a cationic electron or energy transfer quencher.

The bioconjugate cain be represented by the following formula:
D-E-V-D-QSY7′ (SEQ ID NO:7)
wherein “QSY7”′ represents a quencher moiety represented by the following formula: 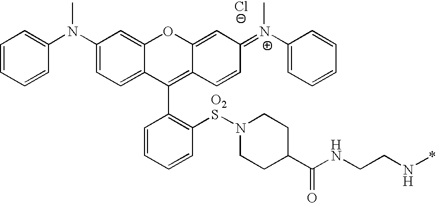
wherein “*” represents the point of attachment of the quencher moiety to the tether. In the peptide substrate represented by SEQ ID NO:7, the quencher moiety is attached to the α-carboxylic acid group of the C-terminal aspartic acid residue via an amino group on the quencher.

The polypeptide tether can be conjugated to the quencher via an amine group on the quencher. An exemplary quencher which can be conjugated to the quencher via an amine group is shown below: 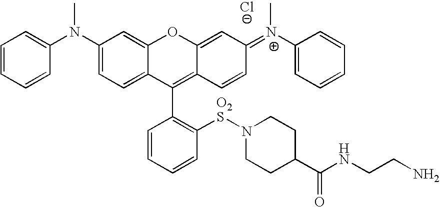
When conjugated to the polypeptide tether via the primary amine group, this quencher forms the QSY7′ quencher moiety. Other quenchers having amine groups can also be used. These quenchers can be synthesized using known chemical synthetic techniques.

The overall charge of the QT bioconjugate represented by SEQ ID NO:7 is −2 (D and E=−1 and QSY7′=+1). The bioconjugate will therefore tend not to associate with a fluorescer having a net negative charge. Cleavage of the tether by the enzyme, however, results in a quencher containing fragment that has an overall positive charge (+1). The quencher containing fragment will therefore tend to associate with a fluorescer having a net negative charge.

According to a further embodiment of the invention, the fluorescer is a virtual polymer comprising an aggregate of donor cyanine dyes and the quencher is an acceptor cyanine dye which, when conjugated to the tether, is unable to form an aggregate with the donor cyanine dyes. The inability of the bioconjugate to form an aggregate with the donor cyanine dyes can be the result of charge effects or steric effects. The acceptor cyanine dye can be a fluorescent molecule or a non-fluorescent molecule. When the acceptor cyanine dye is a fluorescent molecule, the fluorescence of the acceptor can be measured. Alternatively, the fluorescence of the donor can be measured. The assay according to this embodiment of the invention can be an intracellular assay or an extracellular assay.

According to a further embodiment of the invention, a method for assaying for target enzyme activity in a sample is provided which includes: incubating the sample with a bioconjugate comprising a fluorescent dye conjugated to a tether, wherein the tether comprises a segment which can be cleaved by the target enzyme to produce a fluorescent dye containing fragment, and wherein the fluorescent dye containing fragment is capable of forming a dye aggregate which has a different absorption spectra than the bioconjugate; allowing the enzyme to cleave the bioconjugate; and measuring the fluorescence of the sample mixture by exciting the sample at a wavelength wherein the dye aggregate absorbs to a greater extent than the bioconjugate. The amount of fluorescence of the sample mixture indicates the presence and/or amount of target enzyme activity in the sample. The fluorescent dye can be a cyanine molecule. The fluorescent dye containing fragment released from the bioconjugate by enzyme cleavage can be capable of forming a J-aggregate. The target enzyme can be a caspase enzyme. For example, the target enzyme can be caspase-3 and the bioconjugate can have a structure represented by the following formula:

| | |
|---|---|
| D-E-V-D-Cyanine | (SEQ ID NO: 8) |

wherein “Cyanine” represents a cyanine dye moiety.

Exemplary cyanine dye moieties include, but are not limited to, the following:

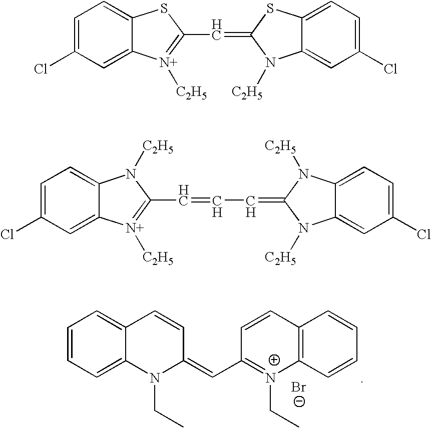

Conventional synthetic chemistry procedures can be used to conjugate the cyanine dyes to the peptide tether. According to one embodiment, the tether can be conjugated to the dye through a functional group on the side chain of the dye. For example, cyanine dyes can be synthesized with side groups having functional groups reactive with groups (i.e., amino and carboxylic acid groups) on the tether. Exemplary of reactive groups which can be synthesized on the dye are amino and carboxylic acid groups. According to one embodiment, an alkyl side group in the cyanine dye can be synthesized with an amino or carboxylic acid group which can then be used for conjugation to a carboxylic acid group on the tether.

An example of a peptide substrate which can be used for protease assays in a J-aggregate format is shown below:

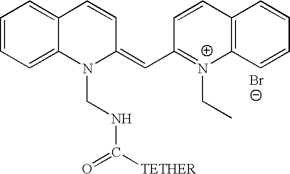

wherein “TETHER” represents a polypeptide capable of being cleaved by a protease enzyme. For caspase-3 enzyme assays, the tether can comprise the following peptide sequence:

| | |
|---|---|
| D-E-V-D | (SEQ ID NO: 9) |

wherein the tether is attached to the dye moiety via the α-carboxylic acid group of the C terminal aspartic acid residue.

Although polypeptide tethers comprising the sequence:

| | |
|---|---|
| D-E-V-D | (SEQ ID NO: 9) |

are disclosed above for caspase-3 enzyme assays, other sequences which can be cleaved by the caspase-3 enzyme can also be used. Further, the above techniques can be used in assays for other protease enzymes wherein the tether comprises a peptide sequence that can be cleaved by the protease enzyme of interest.

According to a further embodiment of the invention, cleavage of the tether by the enzyme results in a dye containing fragment that is capable of forming a J-aggregate. Monitoring for emission from the sample by exciting it at the absorption maximum for the aggregate can be used to provide a signal whose intensity is an indicator of enzyme activity.

Bioconjugates as sot forth above as well as kits comprising the bioconjugates are also provided according to further embodiments of the invention. According to a further embodiment of the invention a kit is provided which includes a fluorescer comprising a plurality of fluorescent species and a bioconjugate comprising a quencher conjugated to a tether, wherein the tether comprises a segment capable of being cleaved by a caspase enzyme. According to this embodiment of the invention, cleavage of the tether results in a quencher containing bioconjugate fragment that has a greater tendency to associate with the fluorescer than the bioconjugate. Further, association of the fluorescer with the quencher results in amplified superquenching of the fluorescer. The plurality of associated fluorescent species can be associated with a solid support. The target caspase enzyme can be caspase-3 and the segment capable of being cleaved by a caspase enzyme can be the peptide sequence:

| | |
|---|---|
| DEVD. | (SEQ ID NO: 9) |

The quencher containing bioconjugate fragment can associate with the fluorescer via coulombic attraction, hydrogen bonding forces, van der waals forces, or covalent bond formation. For example, the fluorescer and the bioconjugate can each have an overall negative charge and the quencher containing bioconjugate fragment can have a net positive charge. The fluorescer can be an anionic conjugate polymer and the quencher can be a cationic electron or energy transfer quencher. The bioconjugate can be a bioconjugate represented by the following formula:

| | |
|---|---|
| D-E-V-D-QSY7′ | (SEQ ID NO: 7) |

wherein “QSY7”′ represents the quencher moiety: 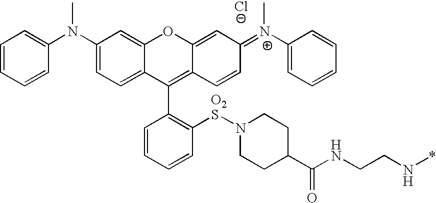
wherein “*” represents the point of attachment of the quencher moiety to the tether and wherein the quencher moiety is conjugated to the tether through the α-carboxylic acid of the c-terminal aspartic acid residue of the tether.

The quencher can be conjugated to the tether through a divalent linker moiety. For example, a quencher having a carboxylic acid group such as QSY7 can be conjugated to the carboxylic acid group of a polypeptide tether using a diamine. Therefore, the divalent linker moiety can be the residue of a diamine. Exemplary diamines include, but are not limited to, diamino alkanes such as 1,2-diaminoethane.

A kit as set forth above is also provided according to a further embodiment wherein the fluorescer is a virtual polymer comprising an aggregate of donor cyanine dyes and the quencher is an acceptor cyanine dye and wherein the acceptor, when conjugated to the tether, is unable to form an aggregate with the donor cyanine dyes. The inability of the bioconjugate to form an aggregate with the donor can be the result of charge effects or steric effects. The acceptor cyanine dye can be a fluorescent molecule or a non-fluorescent molecule.

According to a further embodiment of the invention, a bioconjugate is provided which includes a tether comprising a segment capable of being cleaved by a caspase enzyme and a quencher conjugated to the tether. The caspase enzyme can be caspase-3 and the segment capable of being cleaved by a caspase enzyme can be the peptide sequence:

| | |
|---|---|
| DEVD. | (SEQ ID NO: 9) |

The quencher can be a cationic electron or energy transfer quencher.

The bioconjugate can be a bioconjugate represented by the following formula:

| | |
|---|---|
| D-E-V-D-QSY7′ | (SEQ ID NO: 7) |

wherein “QSY7”′ represents a quencher moiety represented by the following structure: 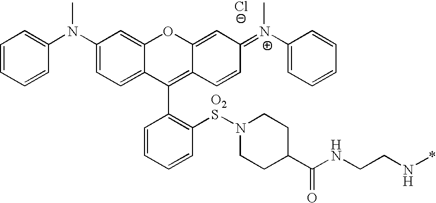
wherein “*” represents the point of attachment of the quencher moiety to the tether and wherein the quencher moiety is conjugated to the tether through the α-carboxylic acid of the c-terminal aspartic acid residue of the tether.

A quencher can be conjugated to the tether through a divalent linker moiety. For example, a quencher having a carboxylic acid group such as QSY7 can be conjugated to the carboxylic acid group of a polypeptide tether using a diamine. Therefore, the divalent linker moiety can be the residue of a diamine. Exemplary diamines include, but are not limited to, diamino alkanes such as 1,2-diaminoethane.

According to a further embodiment of the invention, a bioconjugate is provided which includes a fluorescent dye conjugated to a tether wherein the tether comprises a segment which can be cleaved by the target enzyme to produce a fluorescent dye containing fragment. The fluorescent dye containing fragment is capable of forming a dye aggregate which has a different absorption spectra than the bioconjugate. The fluorescent dye can be a cyanine dye. The fluorescent dye containing fragment can be capable of forming a J-aggregate. The target enzyme can be a caspase enzyme such as caspase-3. The segment capable of being cleaved by the target enzyme can be the peptide sequence:

| | |
|---|---|
| DEVD. | (SEQ ID NO: 9) |

An exemplary bioconjugate has a structure represented by the following formula:

| | |
|---|---|
| D-E-V-D-Cyanine | (SEQ ID NO: 8) |

wherein “cyanine” represents a cyanine dye moiety and wherein the cyanine dye moiety is conjugated to the α-carboxylic acid group of the C-terminal aspartic acid residue of the tether. For example, the bioconjugate can have a structure represented by the following formula:

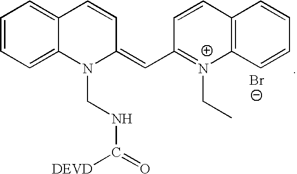

The following prophetic examples (Examples 9 and 10) relate to assays for caspase-3 enzyme activity.

### EXAMPLE 9

### QT Assay Format

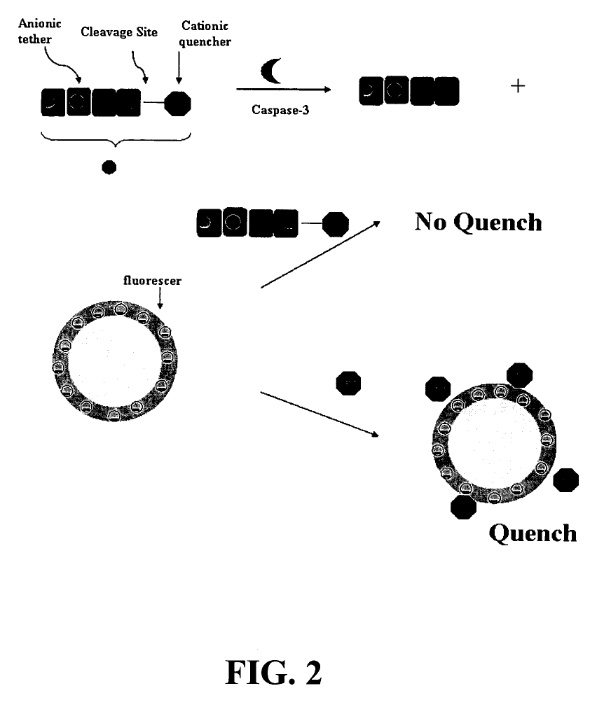

The use of a quencher-tether conjugate in the assay for Caspase-3 enzyme is described in this example. To a known amount of the peptide substrate represented by SEQ ID NO:7 in assay buffer in the well of a 384-well plate is added a sample containing Caspase-3 enzyme. The mixture is allowed to incubate at CRT for a few minutes. The peptide substrate that is overall negatively charged is cleaved by the enzyme to separate the negatively charged peptide and the positively charged quencher. After incubation, the negatively charged polymer represented by formula 2 above is added to the well and the fluorescence from the mixture measured and compared to a control mixture which contains peptide and polymer but no enzyme. The difference in fluorescence between sample and control is a measure of the enzymatic activity in the sample. An assay of this type is represented in FIG. 2.

### EXAMPLE 10

### Aggregate Formation Assay

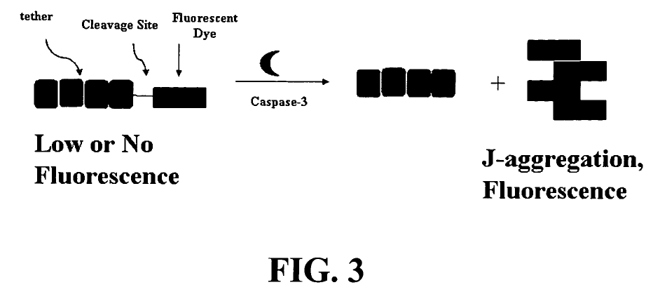

To a known amount of the peptide substrate represented by SEQ ID NO:8 in assay buffer is added a sample solution of Caspase-3 enzyme in one well of a 384-well plate. In another well, the peptide substrate is taken without enzyme. The plate is incubated at CRT and the sample and control wells measured for fluorescence intensity at various time periods. While the control remains unchanged with increasing time, the sample shows increase in fluorescence which is a measure of enzymatic activity. An assay of this type is represented in FIG. 3.

The foregoing description is by way of example only and is not intended to be limiting. Although specific embodiments have been described herein for purposes of illustration, various modifications to these embodiments can be made without the exercise of inventive faculty. All such modifications are within the spirit and scope of the appended claims.

1. A bioconjugate comprising:
a tether comprising a segment capable of recognizing and interacting with β-secretase;
a fluorescer comprising a plurality of fluorescent species, the fluorescer conjugated to a first location on the tether; and
a quencher conjugated to a second location on the tether;
wherein the segment capable of recognizing and interacting with the target biomolecule is located between the first and second locations on the tether, and wherein the plurality of fluorescent species are associated with one another such that the quencher is capable of amplified super-quenching of the fluorescer.

2. The bioconjugate of claim 1, wherein the segment capable of recognizing and interacting with β-secretase comprises the peptide sequence:

| | |
|---|---|
| SEVNLDAEF. | (SEQ ID NO: 1) |

3. The bioconjugate of claim 1, wherein the fluorescer comprises a polymer or oligomer comprising a plurality of fluorescent repeating units.

4. The bioconjugate of claim 1, wherein the fluorescer comprises a solid support associated with a plurality of fluorescent species.

5. The bioconjugate of claim 4, wherein one or more quenchers are each linked to the solid support through a reactive tether.

6. The bioconjugate of claim 4, wherein the solid support is selected from the group consisting of: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes.

7. The bioconjugate of claim 1, wherein the fluorescer is selected from the group consisting of: conjugated polyelectrolytes; fluorescent proteins; biotinylated conjugated polyelectrolytes- functionalized conjugated oligomers; charged conjugated polymers; uncharged conjugated polymers; conjugated polymer blends; and J-aggregated polymer assembly comprising assembled monomers or oligomers.

8. The bioconjugate of claim 1, wherein the fluorescer is a virtual polymer.

9. The bioconjugate of claim 1, wherein the fluorescer is a poly(L-lysine) polymer or oligomer having cyanine pendant groups.

10. The bioconjugate of claim 1, wherein the fluorescer is constructed from an oligosaccharide.

11. The bioconjugate of claim 4, wherein the fluorescer comprises a fluorescent polymer or oligomer.

12. The bioconjugate of claim 11, wherein the fluorescent polymer or oligomer is associated with the solid support by: covalent attachment to the solid support; adsorption onto the surface of the solid support; or by interactions between a biotin moiety on the fluorescent polymer or oligomer and an avidin, neutravidin or streptavidin moiety on the solid support surface.

13. The bioconjugate of claim 1, wherein the fluorescer is conjugated to the tether via a protein molecule.

14. The bioconjugate of claim 13, wherein the protein molecule is selected from the group consisting of: avidin; neutravidin; and streptavidin.

15. The bioconjugate of claim 1, wherein the quencher is non-fluorescent.

16. The bioconjugate of claim 1, wherein the quencher is fluorescent and is capable of reemitting energy absorbed from the fluorescer.

17. A method of assaying for β-secretase activity in a sample comprising:
incubating the sample with a bioconjugate comprising a quencher and a ligand conjugated to a tether at first and second locations respectively, wherein the tether comprises a segment between the first and second locations capable of recognizing and interacting with β-secretase;
adding a fluorescer to the incubated sample to form a sample mixture, the fluorescer comprising a plurality of fluorescent species, wherein the fluorescer comprises a moiety capable of binding the ligand of the bioconjugate such that the bioconjugate can bind to the fluorescer, and wherein binding of the fluorescer to the ligand results in amplified superquenching of the fluorescer;
allowing the ligand on the bioconjugate to bind to the fluorescer; and
subsequently measuring the fluorescence of the sample mixture;
wherein the amount of fluorescence of the sample mixture indicates the presence and/or amount of β-secretase activity in the sample.

18. The method of claim 17, wherein the plurality of associated fluorescent species are associated with a solid support.

19. The method of claim 17, further comprising:
adding the fluorescer to a second sample that contains the bioconjugate but has not been incubated with the enzyme to form a control;
measuring the fluorescence of the control; and
comparing the fluorescence of the control to the fluorescence of the sample mixture;
wherein a difference in the fluorescence between the control and the sample mixture is an indication of the presence and/or the amount of β-secretase in the sample.

20. The method of claim 17, wherein the sample comprises β-secretase and a test compound, the method further comprising:
incubating a second sample containing no test compound with the bioconjugate;
adding the fluorescer to the incubated second sample to form a control;
measuring the fluorescence of the control; and
comparing the fluorescence of the control to the fluorescence of the sample mixture;
wherein a difference in the fluorescence between the control and the sample mixture is an indication of the ability of the test compound to inhibit β-secretase activity in the sample.

21. The method of claim 17, wherein the ligand is a biotin moiety and the moiety on the fluorescer is avidin, neutravidin or streptavidin moiety.

22. A method of assaying for β-secretase activity in a sample, the method comprising:
incubating the sample with a bioconjugate as set forth in claim 1; and
measuring the fluorescence of the incubated sample;
wherein the measured fluorescence of the incubated sample is an indication of the presence and/or the amount of β-secretase activity in the sample.

23. The method of claim 22, further comprising:
measuring the fluorescence of a control; and
comparing the fluorescence of the control to the fluorescence of the incubated sample;
wherein a difference in the fluorescence between the control and the incubated sample is an indication of the presence or amount of β-secretase activity in the sample.

24. The method of claim 22, wherein the sample comprises β-secretase and a test compound and wherein the ability of the test compound to inhibit β-secretase activity is being assayed.

25. The method of claim 22, wherein the fluorescer comprises a solid support and wherein the plurality of fluorescent species are associated with the solid support.

26. The method of claim 25, wherein one or more quenchers are each linked to the solid support through a reactive tether.

27. The method of claim 25, wherein the solid support is selected from the group consisting of: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes.

28. A kit comprising:
a fluorescer comprising a plurality of fluorescent species; and
a bioconjugate comprising a quencher and a ligand conjugated to a tether at first and second locations respectively, wherein the tether comprises a segment between the first and second locations capable of recognizing and interacting with β-secretase;
wherein the fluorescer comprises a moiety capable of binding the ligand on the bioconjugate and wherein the plurality of fluorescent species are associated with one another such that the quencher is capable of amplified superquenching of the fluorescer when the ligand is bound to the fluorescer.

29. The kit of claim 28, wherein the plurality of associated fluorescent species are associated with a solid support.

30. The kit of claim 28, wherein ligand is a biotin moiety.

31. The kit of claim 28, wherein the moiety capable of binding the ligand is selected from the group consisting of avidin, neutravidin and streptavidin.

32. The kit of claim 28, wherein the segment capable of recognizing and interacting with β-secretase comprises the peptide sequence: SEVNLDAEF (SEQ ID NO:1).

33. A bioconjugate comprising:
a tether comprising a segment capable of recognizing and interacting with β-secretase;
a quencher conjugated to a first location on the tether, the quencher capable of quenching the fluorescence of a fluorescer comprising a plurality of associated fluorescent species; and
a biotin molecule conjugated to a second location on the quencher;
wherein the segment capable of recognizing and interacting with the target biomolecule is located between the first and second locations on the tether and wherein the plurality of fluorescent species are associated with one another such that the quencher is capable of amplified quenching of the fluorescer.

34. The bioconjugate of claim 33, wherein the segment capable of recognizing and interacting with β-secretase comprises a polypeptide having the sequence:

| | |
|---|---|
| SEVNLDAEF. | (SEQ ID NO: 1) |

35. The bioconjugate of claim 34, wherein the bioconjugate has a structure represented by:

| | |
|---|---|
| (QSY-7)-TEEISEVNLDAEFK- | (SEQ ID NO: 2) |
| (Nε-Biotin); | |
| (QSY-7)-TEEISEVNLDAEFK- | (SEQ ID NO: 3) |
| (Nε-PEG-Biotin); | |
| (AZO)-TEEISEVNLDAEFK- | (SEQ ID NO: 4) |
| (Nε-Biotin); or | |
| (AZO)-TKKISEVNLDAEFRK- | (SEQ ID NO: 5) |
| (Nε-Biotin); | |

wherein QSY-7, AZO, Biotin and PEG-Biotin represent moieties having the following structures: 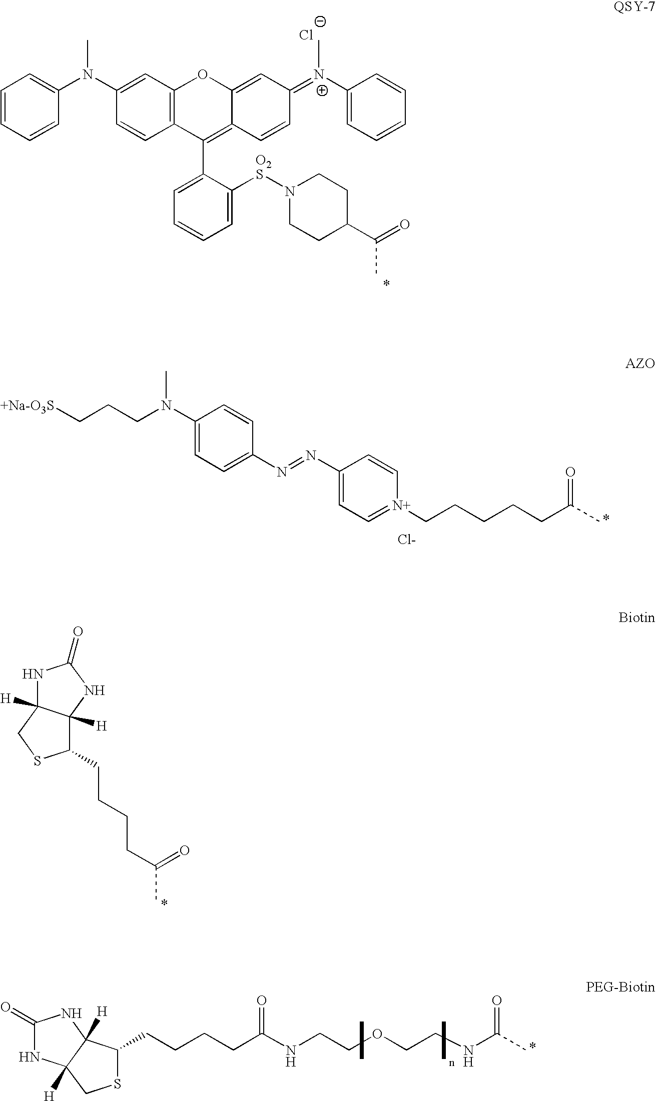
wherein “*” denotes the point of attachment of each moiety to the polypeptide, “Nε” denotes linkage of the biotin moiety or PEG-Biotin moiety to the lysine residue of the polypeptide through the &-amino group of the C-terminal lysine residue, and wherein the QSY-7 and AZO moieties are attached to the polypeptide through the amino group of the N-terminal threonine residue.

36. A method for assaying for target enzyme activity in a sample comprising:
incubating the sample with a bioconjugate comprising a quencher conjugated to a tether, wherein the tether comprises a segment capable of being cleaved by the target enzyme;
adding a fluorescer to the incubated sample to form a sample mixture, the fluorescer comprising a plurality of fluorescent species associated with one another such that a association of the fluorescer with the quencher results in amplified superquenching of the fluorescer; and
allowing the target enzyme to cleave the tether, wherein cleavage of the tether results in a quencher containing bioconjugate fragment that has a greater tendency to associate with the fluorescer than the bioconjugate; and subsequently measuring fluorescence of the sample mixture;
wherein the amount of fluorescence of the sample mixture indicates the presence and/or amount of the target enzyme activity in the sample.

37. The method of claim 36, wherein the target enzyme is a caspase enzyme.

38. The method of claim 36, wherein the target enzyme is caspase-3.

39. The method of claim 38, wherein the segment capable of being cleaved by the target enzyme is the peptide sequence:

| | |
|---|---|
| DEVD. | (SEQ ID NO: 9) |

40. The method of claim 36, wherein association between the quencher containing bioconjugate fragment and the fluorescer comprises coulombic attraction, hydrogen bonding forces, van der waals forces, or covalent bond formation.

41. The method of claim 36, wherein the fluorescer and the bioconjugate each have an overall negative charge and the quencher containing bioconjugate fragment has a net positive charge.

42. The method of claim 41, wherein the fluorescer is an anionic conjugate polymer.

43. The method of claim 41, wherein the quencher is a cationic electron or energy transfer quencher.

44. The method of claim 41, wherein the bioconjugate is represented by the following formula:

| | |
|---|---|
| D-E-V-D-QSY7′ | (SEQ ID NO: 7) |

wherein “QSY7”′ represents a quencher moiety represented by the following structure: 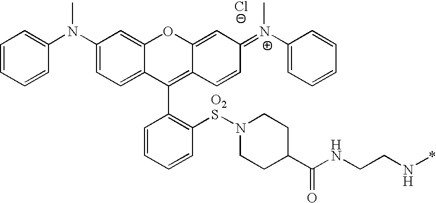
wherein “*” represents the point of attachment of the quencher moiety to the tether and wherein the quencher moiety is conjugated to the tether through the α-carboxylic acid of the c-terminal aspartic acid residue.

45. The method of claim 36, wherein the quencher is conjugated to the tether via a divalent linker.

46. The method of claim 45, wherein the divalent linker is a diamine.

47. The method of claim 36, wherein the fluorescer is a virtual polymer comprising an aggregate of donor cyanine dyes and the quencher is an acceptor cyanine dye and wherein the acceptor, when conjugated to the tether, is unable to form an aggregate with the donor cyanine dyes.

48. The method of claim 47, wherein the inability of the bioconjugate to form an aggregate with the donor is the result of charge effects or steric effects.

49. The method of claim 47, wherein the acceptor cyanine dye is fluorescent.

50. The method of claim 47, wherein the acceptor cyanine dye is fluorescent and wherein the fluorescence of the acceptor is measured.

51. The method of claim 47, wherein the fluorescence of the donor cyanine dye is measured.

52. The method of claim 47, wherein the assay is an intracellular assay or an extracellular assay.

53. A method for assaying for target enzyme activity in a sample comprising:
incubating the sample with a bioconjugate comprising a fluorescent dye conjugated to a tether, wherein the tether comprises a segment capable of being cleaved by the target enzyme to produce a fluorescent dye containing fragment, and wherein the fluorescent dye containing fragment is capable of forming a dye aggregate which has a different absorption spectra than the bioconjugate and;
allowing the target enzyme to cleave the bioconjugate; and
measuring the fluorescence of the sample mixture by exciting the sample at a wavelength wherein the dye aggregate absorbs to a greater degree than the bioconjugate;
wherein the amount of fluorescence of the sample mixture indicates the presence and/or amount of target enzyme activity in the sample.

54. The method of claim 53, wherein the fluorescent dye is a cyanine dye.

55. The method of claim 54, wherein the fluorescent dye containing fragment is capable of forming a J-aggregate.

56. The method of claim 53, wherein the target enzyme is a caspase enzyme.

57. The method of claim 53, wherein the target enzyme is caspase-3.

58. The method of claim 53, wherein the segment capable of being cleaved by the target enzyme is the peptide sequence:

| | |
|---|---|
| DEVD. | (SEQ ID NO: 9) |

59. The method of claim 58, wherein the bioconjugate has a structure represented by the following formula:

| | |
|---|---|
| D-E-V-D-Cyanine | (SEQ ID NO: 8) |

wherein “cyanine” represents a cyanine dye moiety and wherein the cyanine dye moiety is conjugated to the α-carboxylic acid group of the C-terminal aspartic acid residue.

60. The method of claim 59, wherein the bioconjugate has a structure represented by the following formula:

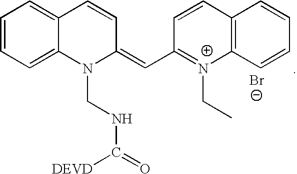

61. A kit comprising:
a fluorescer comprising a plurality of fluorescent species; and
a bioconjugate comprising a quencher conjugated to a tether, wherein the tether comprises a segment capable of being cleaved by a caspase enzyme;
wherein cleavage of the tether results in a quencher containing bioconjugate fragment that has a greater tendency to associate with the fluorescer than the bioconjugate and wherein association of the fluorescer with the quencher results in amplified superquenching of the fluorescer.

62. The kit of claim 61, wherein the plurality of associated fluorescent species are associated with a solid support.

63. The kit of claim 61, wherein the caspase enzyme is caspase-3.

64. The kit of claim 63, wherein the segment capable of being cleaved by a caspase enzyme is the peptide sequence:

| | |
|---|---|
| DEVD. | (SEQ ID NO: 9) |

65. The kit of claim 61, wherein the quencher containing bioconjugate fragment associates with the fluorescer via coulombic attraction, hydrogen bonding forces, van der waals forces or covalent bond formation.

66. The kit of claim 61, wherein the fluorescer and the bioconjugate each have an overall negative charge and the quencher containing bioconjugate fragment has a net positive charge.

67. The kit of claim 66, wherein the fluorescer is an anionic conjugate polymer.

68. The kit of claim 66, wherein the quencher is a cationic electron or energy transfer quencher.

69. The kit of claim 61, wherein the bioconjugate is represented by the following formula:

| | |
|---|---|
| D-E-V-D-QSY7′ | (SEQ ID NO: 7) |

wherein “QSY7”′ represents a quencher moiety represented by the following structure: 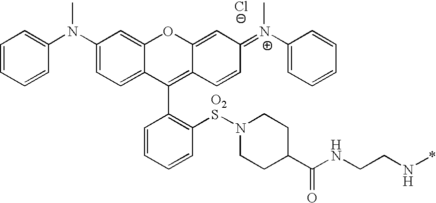
wherein “*” represents the point of attachment of the quencher moiety to the tether and wherein the quencher moiety is conjugated to the tether through the α-carboxylic acid of the c-terminal aspartic acid residue of the tether.

70. The kit of claim 61, wherein the quencher is conjugated to the tether via a divalent linker.

71. The kit of claim 70, wherein the divalent linker is a diamine.

72. The kit of claim 61, wherein the fluorescer is a virtual polymer comprising an aggregate of donor cyanine dyes and the quencher is an acceptor cyanine dye and wherein the acceptor, when conjugated to the tether, is unable to form an aggregate with the donor cyanine dyes.

73. The kit of claim 72, wherein the inability of the bioconjugate to form an aggregate with the donor is the result of charge effects or steric effects.

74. The method of claim 72, wherein the acceptor cyanine dye is a non-fluorescent molecule or a fluorescent molecule capable of reemitting energy absorbed from the fluorescer.

75. A bioconjugate comprising:
a tether comprising a segment capable of being cleaved by a caspase enzyme; and
a quencher conjugated to the tether.

76. The bioconjugate of claim 75, wherein the caspase enzyme is caspase-3.

77. The bioconjugate of claim 76, wherein the segment capable of being cleaved by a caspase enzyme is the peptide sequence:

| | |
|---|---|
| DEVD. | (SEQ ID NO: 9) |

78. The bioconjugate of claim 75, wherein the quencher is a cationic electron or energy transfer quencher.

79. The bioconjugate of claim 75, wherein the bioconjugate is represented by the following formula:

| | |
|---|---|
| D-E-V-D-QSY7′ | (SEQ ID NO: 7) |

wherein “QSY7”′ represents a quencher moiety represented by the following structure: 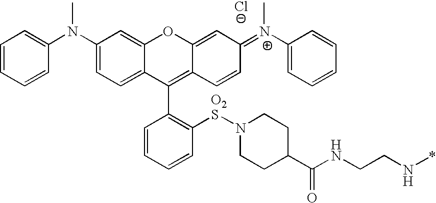
wherein “*” represents the point of attachment of the quencher moiety to the tether and wherein the quencher moiety is conjugated to the tether through the α-carboxylic acid of the c-terminal aspartic acid residue of the tether.

80. The bioconjugate of claim 75, wherein the quencher moiety is conjugated to the tether through a divalent linker.

81. The bioconjugate of claim 80, wherein the divalent linker is a diamine.

82. The bioconjugate of claim 75, wherein the quencher is an acceptor cyanine dye.

83. The bioconjugate of claim 82, wherein the acceptor cyanine dye is fluorescent.

84. A bioconjugate comprising:
a fluorescent dye conjugated to a tether, wherein the tether comprises a segment which can be cleaved by the target enzyme to produce a fluorescent dye containing fragment, and wherein the fluorescent dye containing fragment is capable of forming a dye aggregate which has a different absorption spectra than the bioconjugate.

85. The bioconjugate of claim 84, wherein the fluorescent dye is a cyanine dye.

86. The bioconjugate of claim 84, wherein the fluorescent dye containing fragment is capable of forming a J-aggregate.

87. The bioconjugate of claim 84, wherein the target enzyme is a caspase enzyme.

88. The bioconjugate of claim 84, wherein the target enzyme is caspase-3.

89. The bioconjugate of claim 88, wherein the segment capable of being cleaved by the target enzyme is the peptide sequence:

| | |
|---|---|
| DEVD. | (SEQ ID NO:9) |

90. The bioconjugate of claim 89, wherein the bioconjugate has a structure represented by the following formula:

| | |
|---|---|
| D-E-V-D-Cyanine | (SEQ ID NO:8) |

wherein “cyanine” represents a cyanine dye moiety and wherein the cyanine dye moiety is conjugated to the α-carboxylic acid group of the C-terminal aspartic acid residue of the tether.

91. The bioconjugate of claim 90, wherein the bioconjugate has a structure represented by the following formula: [chemical structure]
